(19) United States
(12) Patent Application Publication
(10) Pub. No.: US 2014/0302138 A1
(43) Pub. Date: Oct. 9, 2014

(54) EXTENDED RELEASE PHARMACEUTICAL COMPOSITIONS CONTAINING CARBAMAZEPINE

(71) Applicant: MICRO LABS LIMITED, Bangalore (IN)

(72) Inventors: Rajesh Kshirsagar, Bangalore (IN); Ganesh Shinde, Bangalore (IN); Amit Kandikurwar, Bangalore (IN)

(21) Appl. No.: 14/351,537

(22) PCT Filed: Oct. 13, 2012
(86) PCT No.: PCT/IB2012/002043

§ 371 Date: Apr. 11, 2014

(30) Foreign Application Priority Data
Oct. 14, 2011 (IN) 3543/CHE/2011

(51) Int. Cl. A61K 9/28; A61K 31/55

(52) U.S. Cl. CPC A61K 9/2866; A61K 31/55; A61K 9/2853
USPC 424/465; 514/217

(57) ABSTRACT

An extended release matrix tablet for once daily administration comprising Carbamazepine or a pharmaceutically acceptable salt thereof and one or more pharmaceutical excipients and process for preparing the same and is bioequivalent to FDA approved Carbamazepine extended release tablet formulations (TEGRETOL®-XR).

### TECHNICAL FIELD OF THE INVENTION

The present invention relates to an extended release pharmaceutical composition for once daily administration comprising Carbamazepine or pharmaceutically acceptable salts thereof and one or more pharmaceutical excipients and process for preparing the same.

### BACKGROUND OF THE INVENTION

Carbamazepine has the chemical name 5H-dibenzo[b,f]azepine-5-carboxamide. Carbamazepine is practically insoluble in water, soluble in alcohol and in acetone. Presently Carbamazepine is available as TEGRETOL XR Extended-Release Tablets in 100 mg, 200 mg and 400 mg strengths. It utilizes osmotic pressure to deliver Carbamazepine at a controlled rate. It has a core of Carbamazepine, and hydroxypropylmethylcellulose, mannitol as an osmotic driving agent, two different grades of hydroxyethyl cellulose (in a 1:1 weight ratio) as the core matrix polymers, lubricant and wetting agent; and a semipermeable wall with a bore connecting the core and the outer environment to release the Carbamazepine.

Oral osmotic dosage forms of Carbamazepine disclosed in U.S. Pat. No. 6,534,090, US 2003/008006 which discloses dosage form which shows ascending rate of release over an extended period.

There are various disadvantages associated with osmotic drug-release technology; such as this technology requires highly sophisticated equipments for processes like compression, coating and laser drilling. Further osmotic drug-release technology requires special excipients like osmogen, osmopolymer, polymer for semipermeable membrane, which ultimately increases cost of manufacturing. Also while preparing osmotic dosage forms using laser drilling the drilling may not performed and such faulty dosage form may not able to release active at all.

Thus, there is still unmet need to develop a simple, stable, extended release solid oral pharmaceutical composition of Carbamazepine, which does not require highly precise technique like drilling on the dosage form and which can provide compositions, which are simple to manufacture, cost effective with stable compositions and acceptable dissolution profile.

### SUMMARY OF THE INVENTION

In one aspect, the present invention provides an extended release pharmaceutical composition comprising Carbamazepine or pharmaceutically acceptable salts thereof and one or more pharmaceutical excipients for once daily dosing.

In yet another aspect the present invention provides a process for preparation of extended release pharmaceutical composition comprising Carbamazepine or pharmaceutically acceptable salts thereof and one or more pharmaceutical excipients for once daily dosing.

In one aspect, the present invention provides an extended release pharmaceutical composition comprising a matrix core comprising of Carbamazepine or pharmaceutically acceptable salts thereof, one or more pharmaceutically acceptable excipients and a coating comprising at least one hydrophobic release controlling agent and at least one hydrophilic release controlling agent for once daily dosing.

In yet another aspect the present invention provides an extended release pharmaceutical composition comprising Carbamazepine or pharmaceutically acceptable salts thereof and one or more pharmaceutical excipients for once daily dosing which is bioavailable and effective with sufficient shelf-life, good pharmaceutical properties, enhancing patient compliance and reducing possible side effects.

In yet another aspect of the present invention provides an extended release pharmaceutical composition comprising Carbamazepine or pharmaceutically acceptable salts and one or more pharmaceutical excipients for once daily dosing, which can be prepared in dosage forms of different strength by proportionally adjusting the quantities of the excipients and the active ingredient, thereby providing a pharmaceutical linearity, without affecting the dissolution profile and bioavailability of the active ingredient.

### BRIEF DESCRIPTION OF THE DRAWING

FIG. 1 Graphical Presentation of dissolution profile of Formulation I, J, K of 400 mg strength and Tegretol XR 400 mg in water.

FIG. 2 Graphical Presentation of dissolution profile of Formulation J of 400 mg strength in 0.1N HCl, pH 4.5 Acetate Buffer and pH 6.8 Phosphate Buffer.

FIG. 3 Graphical Presentation of dissolution profile of Formulation B, D, G of 200 mg strength, Formulation C and H of 100 mg strength and reference product Tegretol XR 200 mg, Tegretol XR 100 mg in water.

FIG. 4 Graphical Presentation of dissolution profile of Formulation D, E, F of 200 mg strength and reference product Tegretol XR 200 mg in 0.1N HCl followed by pH 6.8 phosphate buffer.

FIG. 5 Graphical Presentation of dissolution profile of Tegretol XR 400 mg and formulation L of 400 mg strength in water, 0.1N HCl, pH 4.5 and pH 6.8 at 100 RPM.

FIG. 6 Graphical Presentation of dissolution profile of Tegretol XR 400 mg and formulation L 400 mg strength in water, 0.1N HCl, pH 4.5 and pH 6.8 at 50 RPM.

FIG. 7 Graphical presentation of Dissolution comparison of formulation L at 100 RPM V/S formulation L of 400 mg strength at 50 RPM in water, 0.1N HCl, pH 4.5, pH 6.8.

FIG. 8 Graphical presentation of Dissolution comparison of Tegretol XR 400 mg and formulation L of 400 mg strength at 100 RPM as per USP in CDP Multimedia 1800 ml, Apparatus USP Type I (Basket), at 37±0.5° C.

FIG. 9 Graphical presentation of stability dissolution profile of Tegretol XR 400 mg and formulation L of 400 mg strength at 100 RPM, 1800 ml water, Apparatus USP Type I (Basket), at 37±0.5° C.

FIG. 10 Graphical presentation of comparative Dissolution Profile of Tegretol XR 400 mg v/s Formulation L of 400 mg strength in 0.1 N HCl followed by pH 6.8 Phosphate Buffer.

FIG. 11 Graphical presentation of linear mean plot of Carbamazepine plasma concentration Vs Time for all subjects under fed condition (n=13).

### DETAILED DESCRIPTION OF THE INVENTION

The term “extended release” herein refers to any formulation or dosage form that comprises an active drug and which is formulated to provide a longer duration of pharmacological response after administration of the dosage form than is ordinarily experienced after administration of a corresponding immediate release formulation comprising the same drug in the same amount. Controlled release formulations include, inter alia, those formulations described elsewhere as “controlled release”, “delayed release”, “sustained release”, “prolonged release”, “programmed release”, “time release” and/or “rate controlled” formulations or dosage forms. Further for the purposes of this invention refers to release of an active pharmaceutical agent over a prolonged period of time, such as for example over a period of 8, 12, 16 or 24 hours. By “pharmaceutically acceptable” is meant a carrier comprised of a material that is not biologically or otherwise undesirable.

The term “Carbamazepine” as used in the invention is meant to cover Carbamazepine in the form of freebase or its pharmaceutically acceptable salt(s), hydrate(s), solvate(s) and physiologically functional derivative(s) and precursors thereof. The term also includes all polymorphic forms, whether crystalline or amorphous.

“Cₘₐₓ” as used herein, means maximum plasma concentration of Carbamazepine, produced by the oral administration of the composition of the invention or the immediate release (IR) comparator.

“AUC₀₋ₜ” as used herein, means area under the plasma concentration-time curve, as calculated by the trapezoidal rule over the complete dosing interval for the formulation.

“AUC0-∞” as used herein, means area under the plasma concentration-time curve, as calculated by the trapezoidal rule from time zero to time infinity (AUC_{0-∞}), where AUC_{0-∞}=AUC₀₋ₜ+Ct/λz, Ct is the last measurable drug concentration and λz is the terminal or elimination rate constant calculated according to an appropriate method.

The various embodiments of the present invention can be assembled in several different ways.

In a preferred embodiment, a coated matrix extended release pharmaceutical composition comprising Carbamazepine or pharmaceutically acceptable salts thereof and one or more pharmaceutical excipients for once daily is in the form of a tablet. The core of the coated extended release tablet composition comprises Carbamazepine or pharmaceutically acceptable salts thereof and one or more pharmaceutical excipients

In yet another embodiment the present invention provides an extended release pharmaceutical composition suitable for once daily dosing comprising Carbamazepine or pharmaceutically acceptable salt, derivative, prodrug, metabolite and polymorph thereof and pharmaceutically acceptable excipient having a in-vitro dissolution rate when measured using the USP Type I (Basket apparatus) at 100 rpm in 1800 ml, 0.1N hydrochloric acid for first 2 hours followed by the media with pH 6.8 phosphate buffer at 37° C.±0.5° C. from about 5 to about 25% Carbamazepine released after 1 hour; from about 10 to about 45% Carbamazepine released after 4 hours; from about 35 to about 70% Carbamazepine released after 8 hours; from about 55 to about 78% Carbamazepine released after 12 hours; from about 70 to about 78% Carbamazepine released after 16 hours; and greater than 78% Carbamazepine released after 24 hours.

In yet another embodiment the present invention provides an extended release pharmaceutical composition suitable for once daily dosing comprising Carbamazepine or pharmaceutically acceptable salt, derivative, prodrug, metabolite and polymorph thereof and one or more pharmaceutically acceptable excipients so that upon oral administration the maximum concentrations (Cₘₐₓ) of Carbamazepine in plasma are statistically significantly similar to the reference, and area under the plasma concentration-time curve (AUC) and the minimum plasma concentration are maintained over 24 hours.

In yet another embodiment the present invention provides method of treating epilepsy as well as trigeminal neuralgia comprising administering an extended release, pharmaceutical composition suitable for once daily dosing comprising Carbamazepine or pharmaceutically acceptable salt, derivative, prodrug, metabolite and polymorph thereof as an active ingredient, and one or more pharmaceutically acceptable excipients.

The pharmaceutical compositions according to present invention will, in general comprise of one or more excipients. Examples of pharmaceutical excipients include, but are not limited to binders, fillers or diluents, lubricants, glidants, disintegrants. A combination of excipients may also be used. The amount of excipient(s) employed will depend upon how much active agent is to be used. One excipient can perform more than one function.

Binders include, but are not limited to, starches such as potato starch, wheat starch, corn starch; microcrystalline cellulose such as products known under the registered trade marks Avicel, Filtrak, Heweten or Pharmacel; celluloses such as hydroxypropyl cellulose, hydroxyethyl cellulose, hydroxypropylmethylcellulose (HPMC), ethyl cellulose, sodium carboxymethylcellulose; natural gums like acacia, alginic acid, guar gum; liquid glucose, dextrin, povidone, syrup, polyethylene oxide, polyvinylpyrrolidone, poly-N-vinyl amide, polyethylene glycol, gelatin, poly propylene glycol, tragacanth, combinations there of and other materials known to one of ordinary skill in the art and mixtures thereof.

Fillers or diluents, which include, but are not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants may be selected from, but are not limited to, those conventionally known in the art such as Mg, Al or Ca or Zn stearate, polyethylene glycol, glyceryl behenate, mineral oil, sodium stearyl fumarate, stearic acid, hydrogenated vegetable oil and talc.

Glidants include, but are not limited to, silicon dioxide; magnesium trisilicate, powdered cellulose, starch, talc and tribasic calcium phosphate, calcium, silicate, magnesium silicate, colloidal silicon dioxide, silicon hydrogel and other materials known to one of ordinary skill in the art.

The formulation according to present invention may also comprise a disintegrant which may be included in all or part of the oral dosage form to ensure rapid disintegration of the dosage form or part of the dosage form (for example, one of the layers in a bilayer tablet) after administration.

Disintegrants include, but are not limited to: alginic acid, carboxymethylcellulose calcium, carboxymethylcellulose sodium, croscarmellose sodium, crospovidone, guar gum, magnesium aluminium silicate, sodium alginate, sodium starch glycolate and starches and other materials known to one of ordinary skill in the art and combinations thereof.

It should be appreciated that there is considerable overlap between the above-listed additives in common usage, since a given additive is often classified differently by different practitioners in the field, or is commonly used for any of several different functions. Thus, the above-listed additives should be taken as merely exemplary, and not limiting, of the types of additives that can be included in compositions of the present invention. One or more of these additives can be selected and used by the skilled artisan having regard to the particular desired properties of the dosage form by routine experimentation without any undue burden.

The amount of each type of additive employed may vary within ranges conventional in the art.

In a preferred embodiment, the core matrix tablet of the present invention is formulated with Carbamazepine or pharmaceutically acceptable salts thereof, a matrix forming polymer, a diluent, a binder and a lubricant.

The tablets comprising Carbamazepine or pharmaceutically acceptable salts thereof can be prepared by processes well known to those of skill in the art. For example, core tablets can be prepared by wet granulation, dry granulation, melt granulation and the like. In a preferred embodiment, the core tablets comprising Carbamazepine or pharmaceutically acceptable salts thereof are prepared by wet granulation.

In a further embodiment, the tablets are prepared by melt granulation. The matrix tablet core comprising Carbamazepine or pharmaceutically acceptable salts thereof are then coated with a suitable rate controlling composition to control the release rate of Carbamazepine or pharmaceutically acceptable salts thereof. The rate controlling composition can comprise one or more hydrophobic agents and hydrophilic agents.

Suitable hydrophobic agents include, but are not limited to polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly (ethyl methacrylate), poly(butyl methacrylate), poly(isobutyl methacrylate), and poly (hexyl methacrylate), poly(isodecyl methacrylate), poly(lauryl methacrylate), poly (phenyl methacrylate), poly(methyl acrylate), poly(isopropyl acrylate), -poly (isobutyl acrylate), poly(octadecyl acrylate), waxes such as beeswax, carnauba wax, paraffin wax, microcrystalline wax, and, ozokerite; fatty alcohols such as cetostearyl alcohol, stearyl alcohol, cetyl alcohol and myristyl alcohol, and fatty acid esters such as glyceryl monostearate; glycerol monooleate, acetylated monoglycerides, tristearin, tripalmitin, cetyl esters wax, glyceryl palmitostearate, glyceryl behenate, and hydrogenated vegetable oils and the like.

Suitable hydrophilic agents include, but are not limited to water soluble polymers such as hydroxyethyl cellulose, hydroxypropyl cellulose, carboxymethyl cellulose, hydroxypropylmethyl cellulose, sodium carboxymethyl cellulose, vinylpyrrolidone/vinyl acetate copolymer for example marketed as Plasdone® S-630, polyvinyl alcohol, polyethylene glycol and the like, Saccharides such as monosaccharides, disaccharides, oligosaccharides, polysaccharides or sugar alcohols which include but are not limited to sucrose, xylitol, mannitol, sorbitol, glucose, fructose, galactose, maltitol, lactose, maltodextrin, Water soluble organic acids, water soluble salts of organic acids, water soluble organic bases, water soluble salts of organic bases which include but are not limited to citric acid or salts thereof, amino acids or salt thereof, inorganic salts such as sodium carbonate, sodium bicarbonate, potassium chloride and sodium chloride and the like.

Suitable matrix forming agents include, but are not limited to hydroxypropylmethylcellulose, hydroxypropylcellulose, mannitol, dextrates, lactose, dibasic calcium phosphate, microcrystalline cellulose, hydroxyl ethyl cellulose and ethyl cellulose.

In a still preferred embodiment of the present invention, the coating comprises from about 0.1 to 50% w/w of the core matrix tablet, more preferably the coating comprises from about 0.5 to 20% w/w of the core.

The coating composition may optionally contain other excipients, which include, but are not limited to plasticizers, opacifiers, coloring agents and antifoaming agents. Examples of plasticizers include, but are not limited to citrates such as triethylcitrate, acetyl tributyl citrate, phthalates, dibutyl sebacate, triacetin, polyethylene glycol and the like.

Examples of opacifying agents and coloring agents include, but are not limited to titanium dioxide, talc, aluminum lake dyes, insoluble pigments, water-soluble dyes and the like. Antifoaming agents include, but are not limited to silicone, simethicone and the like.

The core tablets can be coated using any of the techniques well known to the persons skilled in the art. In a preferred embodiment, coating of core tablets of Carbamazepine is carried out by spraying aqueous and/or non-aqueous solution/dispersion and its mixtures of the coating composition excipients onto a core tablet bed in a perforated coating pan.

The various embodiments of the present invention can be assembled in several different ways.

In one embodiment, the present invention provides an extended release matrix tablet comprising Carbamazepine or pharmaceutically acceptable salts thereof and one or more pharmaceutical excipients.

In yet another embodiment, the present invention provides an extended release matrix tablet comprising Carbamazepine or pharmaceutically acceptable salts thereof and one or more pharmaceutical excipients and optional coating of one or more hydrophobic release controlling agents and hydrophilic release controlling agents.

In yet another embodiment, the present invention provides an extended release matrix tablet comprising Carbamazepine or pharmaceutically acceptable salts thereof and one or more pharmaceutical excipients wherein the tablet is further coated with a rate controlling composition comprising one or more hydrophobic agents and one or more hydrophilic agents.

In yet another embodiment, the present invention provides a process of preparing extended release matrix tablet comprising Carbamazepine or pharmaceutically acceptable salts thereof and one or more pharmaceutical excipients wherein the process can be selected from direct compression, dry granulation, wet granulation (aqueous/non-aqueous or combination) and melt granulation.

The following examples illustrate preferred embodiments in accordance with the present invention without limiting the scope or spirit of the invention.

### Example 1

Compositions A, B and C having ingredients as provided hereinbelow are prepared.

| | Formulation | | |
|---|---|---|---|
| | A | B | C |
| Ingredients | mg/Tablet | | |
| Carbamazepine | 400.0 | 200.0 | 100.0 |
| Hydroxypropylmethylcellulose | 47.0 | 23.5 | 11.75 |
| Hydroxyethyl Cellulose (Natrosol 250L | 20.0 | 10.0 | 5.0 |
| Pharm) | | | |
| Hydroxyethyl Cellulose (Natrosol 250 HX | 40.0 | 20.0 | 10.0 |
| Pharm) | | | |
| Mannitol | 110.3 | 55.15 | 27.575 |
| Dextrates | 108.2 | 54.10 | 27.05 |
| Sodium Lauryl Sulphate | 5.0 | 2.5 | 1.25 |
| Iron oxide yellow | 0.1 | 0.05 | 0.025 |
| Iron oxide Red | 0.1 | 0.05 | 0.025 |
| Titanium Dioxide | 1.3 | 0.65 | 0.325 |
| Purified water | q.s. | q.s. | q.s. |
| Magnesium Stearate | 8.0 | 4.0 | 2.0 |
| Total weight of core tablet | 740.0 | 370.0 | 185.0 |
| Coating | | | |
| Cellulose Acetate | 18.5 | 9.25 | 4.625 |
| Polyethylene Glycol 400 | 1.85 | 0.925 | 0.4625 |
| Triethylcitrate | 1.85 | 0.925 | 0.4625 |
| Dichloromethane | q.s. | q.s. | q.s. |
| Methanol | q.s. | q.s. | q.s. |
| Total weight of Tablet | 762.2 | 381.1 | 190.55 |

### Manufacturing Procedure:

Carbamazepine, hydroxypropylmethylcellulose, hydroxyethyl cellulose, Mannitol, dextrates, titanium dioxide, sodium lauryl sulphate, iron oxide yellow and iron oxide red were sifted through suitable sieve and mixed in rapid mixer granulator for 10 minutes. This mixture was wet granulated by using purified water in rapid mixture granulator. The wet granules were dried in rapid dryer. The dried granules passed through suitable sieve and mixed with sifted magnesium stearate in blender for 5 minutes. The lubricated blend was compressed into single rotary tablet machine to obtain tablets of 100 mg, 200 mg and 400 mg strengths.

Cellulose acetate, PEG 400 and triethylcitrate were dissolved in mixture of 80 parts methylene chloride and 20 parts methanol. The core tablets of respective strengths were coated in coating pan by using this solution to a desired weight gain.

### Example 2

Composition D having ingredients as provided hereinbelow is prepared.

| | Formulation D |
|---|---|
| Ingredients | mg/Tablet |
| Carbamazepine | 200.0 |
| Hydroxypropylmethylcellulose | 23.5 |
| Hydroxyethyl Cellulose (Natrosol 250L Pharm) | 10.0 |
| Hydroxyethyl Cellulose (Natrosol 250 HX Pharm) | 20.0 |
| Mannitol | 55.15 |
| Dextrates | 54.10 |
| Sodium Lauryl Sulphate | 2.5 |
| Iron oxide yellow | 0.05 |
| Iron oxide Red | 0.05 |
| Titanium Dioxide | 0.65 |
| Purified water | q.s. |
| Magnesium Stearate | 4.0 |
| Total weight of core tablet | 370.0 |
| Coating | |
| Cellulose acetate | 6.17 |
| Polyethylene Glycol 400 | 2.47 |
| TEC | 2.47 |
| Dichloromethane | q.s. |
| Methanol | q.s. |
| Total weight of tablet | 381.11 |

### Manufacturing Procedure:

Carbamazepine, hydroxypropylmethylcellulose, hydroxyethyl cellulose, Mannitol, dextrates, titanium dioxide, sodium lauryl sulphate, iron oxide yellow and iron oxide red were sifted through suitable sieve and mixed. This mixture was wet granulated by using purified water. The wet granules were dried. The dried granules passed through suitable sieve and mixed with sifted magnesium stearate in blender for 5 minutes. The lubricated blend was compressed into single rotary tablet machine to obtain tablets.

The core tablets were coated with coating solution of Cellulose acetate, PEG 400 and triethylcitrate dissolved in mixture of 80 parts methylene chloride and 20 parts methanol to a desired weight gain.

### Example 3

Composition D having ingredients as provided herein below is prepared.

| | Formulation E |
|---|---|
| Ingredients | mg/Tablet |
| Carbamazepine | 200.0 |
| Hydroxypropylmethylcellulose | 23.5 |
| Hydroxyethyl Cellulose (Natrosol 250L Pharm) | 10.0 |
| Hydroxyethyl Cellulose (Natrosol 250 HX Pharm) | 20.0 |
| Mannitol | 55.15 |
| Dextrates | 54.10 |
| Sodium Lauryl Sulphate | 2.5 |
| Iron oxide yellow | 0.05 |
| Iron oxide Red | 0.05 |
| Titanium Dioxide | 0.65 |
| Purified water | q.s. |
| Magnesium Stearate | 4.0 |
| Total weight of core tablet | 370.0 |
| Coating | |
| Cellulose acetate | 3.96 |
| Hydroxypropyl Cellulose | 0.793 |
| Triethylcitrate | 0.793 |
| Dichloromethane | q.s. |
| Methanol | q.s. |
| Total weight of tablet | 375.546 |

### Manufacturing Procedure:

Carbamazepine, hydroxypropylmethylcellulose, hydroxyethyl cellulose, Mannitol, dextrates, titanium dioxide, sodium lauryl sulphate, iron oxide yellow and iron oxide red were sifted through suitable sieve and mixed. This mixture was wet granulated by using purified water. The wet granules were dried. The dried granules passed through suitable sieve and mixed with sifted magnesium stearate in blender for 5 minutes. The lubricated blend was compressed into single rotary tablet machine to obtain tablets.

The core tablets were coated with coating solution of cellulose acetate, hydroxypropyl cellulose and triethylcitrate dissolved in mixture of 80 parts methylene chloride and 20 parts methanol to a desired weight gain.

### Example 4

Composition F having ingredients as provided hereinbelow is prepared.

| | Formulation F |
|---|---|
| Ingredients | mg/Tablet |
| Carbamazepine | 200.0 |
| Hydroxypropylmethylcellulose | 23.5 |
| Hydroxyethyl Cellulose (Natrosol 250L Pharm) | 10.0 |
| Hydroxyethyl Cellulose (Natrosol 250 HX Pharm) | 20.0 |
| Mannitol | 55.15 |
| Dextrates | 54.10 |
| Sodium Lauryl Sulphate | 2.5 |
| Iron oxide yellow | 0.05 |
| Iron oxide Red | 0.05 |
| Titanium Dioxide | 0.65 |
| Purified water | q.s. |
| Magnesium Stearate | 4.0 |
| Total weight of core tablet | 370.0 |
| Coating | |
| Cellulose acetate | 3.96 |
| Hydroxypropyl Cellulose | 0.793 |
| Polyethylene Glycol 400 | 0.793 |
| Dichloromethane | q.s. |
| Methanol | q.s. |
| Total weight of tablet | 375.546 |

### Manufacturing Procedure:

Carbamazepine, hydroxypropylmethylcellulose, hydroxyethyl cellulose, Mannitol, dextrates, titanium dioxide, sodium lauryl sulphate, iron oxide yellow and iron oxide red were sifted through suitable sieve and mixed. This mixture was wet granulated by using purified water. The wet granules were dried. The dried granules passed through suitable sieve and mixed with sifted magnesium stearate in blender for 5 minutes. The lubricated blend was compressed into single rotary tablet machine to obtain tablets.

The core tablets were coated with coating solution of cellulose acetate, hydroxypropyl cellulose and polyethylene glycol 400 dissolved in mixture of 80 parts methylene chloride and 20 parts methanol to a desired weight gain.

### Example 5

Compositions G and H having ingredients as provided hereinbelow are prepared.

| | Formulation | |
|---|---|---|
| | G | H |
| Ingredients | mg/Tablet | |
| Carbamazepine | 200.0 | 100.0 |
| Hydroxypropylmethylcellulose | 23.5 | 11.75 |
| Hydroxyethyl Cellulose (Natrosol 250L Pharm) | 10.0 | 5.0 |
| Hydroxyethyl Cellulose (Natrosol 250 HX | 20.0 | 10.0 |
| Pharm) | | |
| Mannitol | 55.15 | 27.575 |
| Dextrates | 54.75 | 27.375 |
| Sodium Lauryl Sulphate | 2.5 | 1.25 |
| Iron oxide yellow | 0.1 | 0.05 |
| Purified water | q.s. | q.s. |
| Magnesium Stearate | 4.0 | 2.0 |
| Total weight of core tablet | 370.0 | 185 |
| Coating | | |
| Cellulose acetate | 9.00 | 5.29 |
| Polyethylene Glycol 400 | 2.81 | 1.40 |
| Dichloromethane | q.s. | q.s. |
| Methanol | q.s. | q.s. |
| Total weight of tablet | 381.81 | 191.69 |

### Manufacturing Procedure:

Carbamazepine, hydroxypropylmethylcellulose, hydroxyethyl cellulose, Mannitol, dextrates, titanium dioxide, sodium lauryl sulphate and iron oxide yellow were sifted through suitable sieve and mixed in rapid mixer granulator for 10 minutes. This mixture was wet granulated by using purified water. The wet granules were dried. The dried granules passed through suitable sieve and mixed with sifted magnesium stearate in blender for 5 minutes. The lubricated blend was compressed into single rotary tablet machine to obtain tablets of 100 mg and 200 mg.

The core tablets of 100 mg and 200 mg strength were separately coated in coating pan with coating solution of respective quantity of cellulose acetate and polyethylene glycol dissolved in mixture of 80 parts methylene chloride and 20 parts methanol to a desired weight gain.

### Example 6

Compositions I, J and K having ingredients as provided hereinbelow are prepared.

| | Formulation | | |
|---|---|---|---|
| | I | J | K |
| Ingredients | mg/Tablet | | |
| Carbamazepine | 400.0 | 400.0 | 400.0 |
| Hydroxypropylmethylcellulose | 47.0 | 47.0 | 40.0 |
| Hydroxyethyl Cellulose (Natrosol 250L | 20.0 | 20.0 | 10.0 |
| Pharm) | | | |
| Hydroxyethyl Cellulose (Natrosol 250 HX | 40.0 | 40.0 | 20.0 |
| Pharm) | | | |
| Mannitol | 110.3 | 110.3 | 109.0 |
| Dextrates | 109.3 | 109.3 | 108.8 |
| Sodium Lauryl Sulphate | 5.0 | 5.0 | 5.0 |
| Iron oxide yellow | 0.2 | 0.2 | 0.2 |
| Purified water | q.s. | q.s. | q.s. |
| Magnesium Stearate | 8.0 | 8.0 | 7.0 |
| Total weight of core tablet | 740.0 | 740.0 | 700.0 |
| Coating | | | |
| Cellulose acetate | 14.2 | 14.2 | 13.7 |
| Polyethylene Glycol 400 | 5.68 | 11.4 | 8.2 |
| Dichloromethane | q.s. | q.s. | q.s. |
| Methanol | q.s. | q.s. | q.s. |
| Total weight of coated tablet | 759.88 | 765.6 | 721.9 |

### Manufacturing Procedure:

Carbamazepine, hydroxypropylmethylcellulose, hydroxyethyl cellulose, Mannitol, dextrates, titanium dioxide, sodium lauryl sulphate and iron oxide yellow were sifted through suitable sieve and mixed in rapid mixer granulator for 10 minutes. This mixture was wet granulated by using purified water. (For Formulation K granulation was done by dissolving SLS in purified water). The wet granules were dried. The dried granules passed through suitable sieve and mixed with sifted magnesium stearate in blender for 5 minutes. The lubricated blend was compressed into single rotary tablet machine to obtain tablets of 400 mg strength.

The core tablets was coated in coating pan with coating solution of respective quantity of cellulose acetate and polyethylene glycol dissolved in mixture of 80 parts methylene chloride and 20 parts methanol to a desired weight gain.

### Example 7

Composition L having ingredients as provided hereinbelow is prepared.

| | | |
|---|---|---|
| | | Formulation L |
| | | Quantity |
| Sr. No. | Ingredients | mg/Unit |

Granulation

| | | |
|---|---|---|
| 1 | Carbamazepine USP | 400.0 |
| 2 | Hypromellose USP | 40.0 |
| | (Methocel E5 Premium LV) | |
| 3 | Hydroxyethyl Cellulose USPNF | 10.0 |
| | (Natrosol 250L PHARM) | |
| 4 | Hydroxyethyl Cellulose USPNF | 20.0 |
| | (Natrosol 250 HX PHARM) | |
| 5 | Mannitol 35 USPNF (Perlitol 50C) | 109.0 |
| 6 | Dextrates, Hydrated USPNF | 107.8 |
| | (Emdex Non GMO) | |
| 7 | Sodium Lauryl Sulfate USPNF | 5.0 |
| | (TEXAPON K12 P PH) | |
| 8 | Iron Oxide Yellow USPNF | 0.2 |
| 9 | Purified Water | q.s. |

Lubrication

| | | |
|---|---|---|
| 10 | Magnesium Stearate USPNF | 8.0 |
| | Total weight of tablet | 700.0 |

Coating

| | | |
|---|---|---|
| 11 | Cellulose Acetate (398-10) USPNF | 13.72 |
| 12 | Polyethylene Glycol USPNF | 8.23 |
| | (Lutrol E 400) | |
| 13 | Methylene Chloride USPNF | q.s. |
| 14 | Methanol USPNF | q.s. |

| | |
|---|---|
| Total weight of coated tablet | 721.95 |

### Manufacturing Procedure:

Carbamazepine, Hypromellose, Hydroxyethyl cellulose (Natrosol 250L PHARM), Hydroxyethyl cellulose (Natrosol 250 HX PHARM) Mannitol, dextrates, are sifted through #20 mesh sieve and iron oxide yellow through #80 mesh sieve and mixed in RMG for 10 minutes. Sodium lauryl sulfate was dissolved in sufficient purified water. Wet granulation was carried out by using SLS solution as binder. The wet granules were dried. The dried granules passed through suitable sieve and mixed with sifted magnesium stearate in blender for 3 minutes. The lubricated blend was compressed into single rotary tablet machine to obtain tablets. Cellulose acetate and PEG 400 was dissolved in the mixture of Methylene chloride and methanol and core tablets were coated.

### Example 8

### Pharmacokinetic Study of Extended Release Carbamazepine Tablet

This study was a randomized, open label, balanced, single center, two treatments, two period, two sequences, single dose; crossover bioequivalence study with a 22 days washout between doses.

The two formulations compared were the 400 mg tablet of the formulation of the present invention (treatment A), and Reference product (Tegretol® XR 400 Mg [Carbamazepine extended release tablets] (treatment B)

In total, 14 healthy adult male volunteers (aged 18-45 years and of body mass index within 18.50 to 24.99 kg/m², not weighing less than 50 kg) entered the study. Thirteen subjects completed the study.

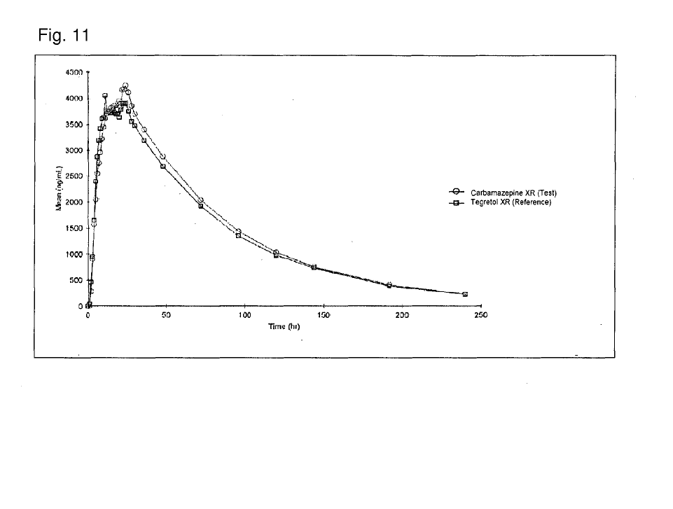

Blood samples were collected at the following times: pre-dose sample was collected within one hour prior to drug administration and the post dose samples were collected at 1, 2, 3, 4, 5, 6, 7, 8, 9, 10, 11, 12, 13, 14, 15, 16, 17, 18, 19, 20, 21, 22, 23, 24, 26, 28, 30, 36, 48, 72, 96, 120, 144, 192 and 240 hours after dosing. Plasma samples were analyzed for Carbamazepine concentrations using a validated LCMS method. The results of the pharmacokinetic analyses are shown in FIG. 11

Under the single dose fed conditions of this study, the formulation of the tablets of the present invention and Tegretol® CR 400 mg were bioequivalent, having comparative rates of absorption and comparative extent of absorption. The ratio of mean plasma concentrations for the formulation of the present invention to Tegretol® XR 400 mg tablets was 106.00%, 105.50% and 96.88% for AUC₀₋ₜ, AUC_{0-∞} and Cₘₐₓ respectively.

The 90% confidence intervals of test Vs Reference were observed as 96.62% to 116.3% for AUC₀₋ₜ, 96.34% to 115.54% for AUC_{0-∞}, and 85.62% to 109.62% for Cₘₐₓ and matching with the regulatory agencies bioequivalence acceptance criteria.

The formulation of the present invention was therefore, found to be bioequivalent to Tegretol® XR 400 mg formulation (Novartis pharma productions GmbH Wehr, Germany). Thus, the formulation of the present invention is clearly suitable and effective for once a day oral administration of Carbamazepine.

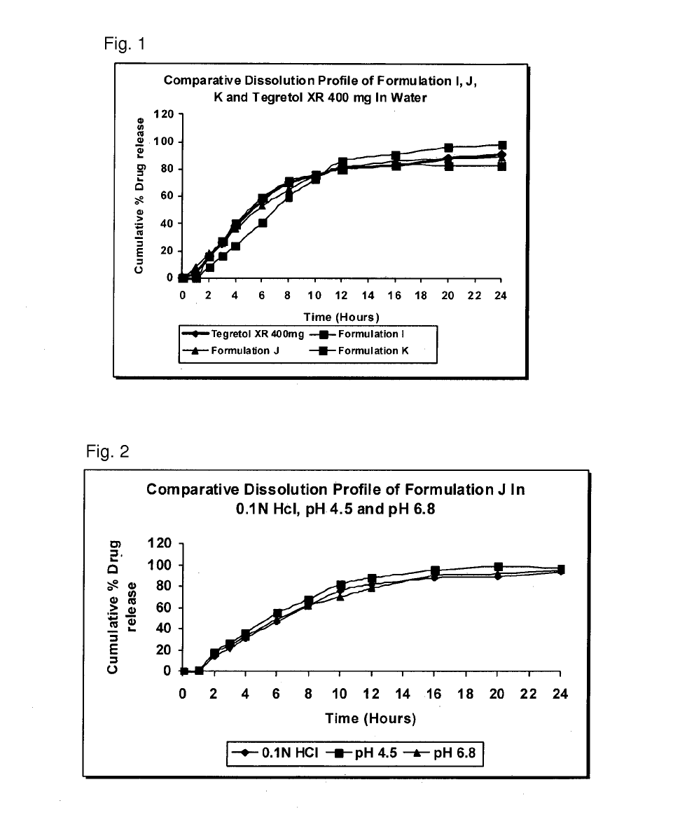

The dissolution of formulation I, J, K of 400 mg strength and reference product Tegretol XR 400 mg was carried out in Water 1800 ml, Apparatus USP Type I (Basket), 100 RPM, at 37±0.5° C., is determined and the dissolution profile is as provided in Table 1 (provided herein below) and a graphical representation of the same is provided in FIG. 1.

TABLE 1

Formulation

| | | | |
|---|---|---|---|
| Tegretol XR 400 mg | I | J | K |

| | |
|---|---|
| Time (hrs) | Cumulative % Drug Released |

| | | | | |
|---|---|---|---|---|
| 1 | 4 | 1 | 8 | 0 |
| 2 | 16 | 8 | 18 | 16 |
| 3 | 26 | 16 | 26 | 27 |
| 4 | 38 | 24 | 36 | 40 |
| 6 | 57 | 41 | 53 | 59 |
| 8 | 70 | 60 | 65 | 71 |
| 10 | 75 | 73 | 75 | 76 |
| 12 | 81 | 86 | 81 | 80 |
| 16 | 83 | 91 | 86 | 83 |
| 20 | 88 | 96 | 88 | 83 |
| 24 | 91 | 98 | 89 | 83 |

Further, the dissolution of formulation J was evaluated in 1800 ml, 0.1N HCl, pH 4.5 Acetate Buffer, and pH 6.8 Phosphate Buffer, Apparatus USP Type I (Basket), 100 RPM, at 37±0.5° C., and the dissolution profile is as provided in Table 2 (herein below) and a graphical representation of the same is provided in FIG. 2.

TABLE 2

| | Formulation J | | |
|---|---|---|---|
| | 0.1N HCl | pH 4.5 | pH 6.8 |
| Time (hrs) | Cumulative % Drug Released | | |
| 1 | 1 | 1 | 1 |
| 2 | 14 | 18 | 17 |
| 3 | 22 | 27 | 25 |
| 4 | 31 | 36 | 33 |
| 6 | 47 | 55 | 49 |
| 8 | 62 | 68 | 62 |
| 10 | 76 | 82 | 70 |
| 12 | 82 | 88 | 78 |
| 16 | 88 | 95 | 90 |
| 20 | 89 | 99 | 92 |
| 24 | 94 | 97 | 95 |

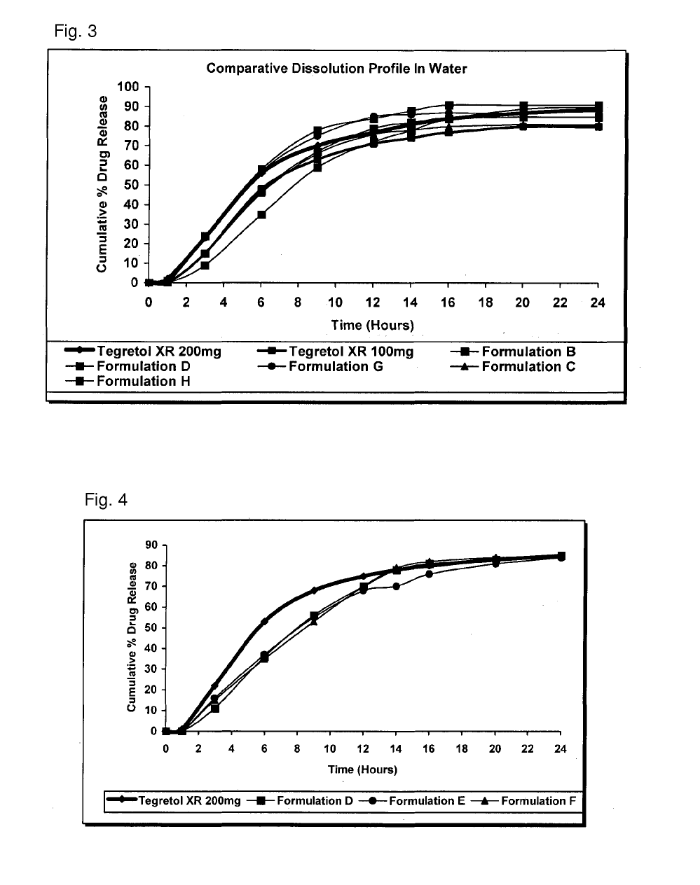

The dissolution of formulation B, D, G of 200 mg strength, Formulation C and H of 100 mg strength and reference product Tegretol XR 200 mg, Tegretol 100 mg was carried out in Water 900 ml, Apparatus USP Type I (Basket), 100 RPM, at 37±0.5° C. is determined and the dissolution profile is as provided in Table 3 (herein below) and a graphical representation of the same is provided in FIG. 3.

TABLE 3

| | | | Formulation | | | | |
|---|---|---|---|---|---|---|---|
| | Tegretol | | B | D | G | C | H |
| | XR | Tegretol | Strength | | | | |
| | 200 mg | XR 100 mg | 200 mg | | | 100 mg | |
| Time (hrs) | % Drug Released | | | | | | |
| 1 | 2 | 0 | 0 | 0 | 0 | 3 | 1 |
| 3 | 23 | 15 | 9 | 15 | 23 | 15 | 24 |
| 6 | 56 | 48 | 35 | 47 | 57 | 46 | 58 |
| 9 | 70 | 63 | 59 | 67 | 75 | 66 | 78 |
| 12 | 77 | 71 | 72 | 79 | 85 | 76 | 84 |
| 14 | 81 | 74 | 78 | 82 | 86 | 78 | 88 |
| 16 | 84 | 77 | 84 | 84 | 87 | 80 | 91 |
| 20 | 87 | 80 | 89 | 85 | 87 | 81 | 91 |
| 24 | 89 | 80 | 90 | 85 | 88 | 81 | 91 |

The dissolution of formulation D, E, F of 200 mg strength and reference product Tegretol XR 200 mg, was carried out in 0.1N HCl followed by pH 6.8 Phosphate buffer, 900 ml, Apparatus USP Type I (Basket), 100 RPM, at 37±0.5° C. is determined and the dissolution profile is provided in Table 4 (herein below) and a graphical representation of the same is provided in FIG. 4.

TABLE 4

Formulation

| | | | | |
|---|---|---|---|---|
| | Tegretol- | | | |
| Time | XR 200 mg | D | E | F |

| | | |
|---|---|---|
| Media | (Hours) | Cumulative % drug released |

| | | | | | |
|---|---|---|---|---|---|
| 0.1N HCl | 1 | 1 | 0 | 0 | 0 |
| pH 6.8, | 3 | 22 | 11 | 16 | 15 |
| Phosphate | 6 | 53 | 36 | 37 | 35 |
| buffer | 9 | 68 | 56 | 55 | 53 |
| | 12 | 75 | 70 | 68 | 70 |
| | 14 | 78 | 78 | 70 | 79 |
| | 16 | 80 | 81 | 76 | 82 |
| | 20 | 83 | 83 | 81 | 84 |
| | 24 | 85 | 85 | 84 | 85 |

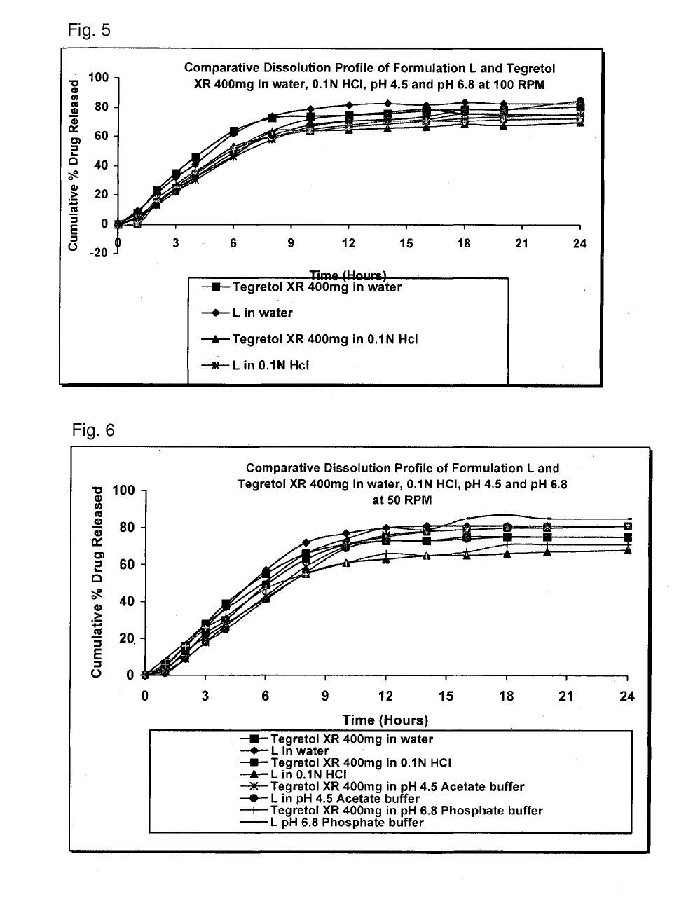

Comparative dissolution profile of Tegretol XR 400 mg (represented as TG in the table 5) and formulation L of 400 mg in CDP Multimedia 1800 ml, Apparatus USP Type I (Basket), 100 RPM, at 37±0.5° C., is determined and the dissolution profile is provided in Table 5 (herein below) and a graphical representation of the same is provided in FIG. 5.

TABLE 5

| | Formulation | | | | | | | |
|---|---|---|---|---|---|---|---|---|
| | TG | L | TG | L | TG | L | TG | L |
| | Media | | | | | | | |
| | | | | | pH 4.5 | | pH 6.8 | |
| | | | | | Acetate | | Phosphate | |
| | Water | | 0.1N HCl | | buffer | | buffer | |
| | Units | | | | | | | |
| | n = 6 | n = 6 | n = 6 | n = 6 | n = 6 | n = 6 | n = 6 | n = 6 |
| | Condition | | | | | | | |
| | 1800 ml, USP Type I (Basket), 100 RPM | | | | | | | |
| Time | Cumulative % Drug Released | | | | | | | |
| 0 | 0 | 0 | 0 | 0 | 0 | 0 | 0 | 0 |
| 1 | 8 | 9 | 5 | 4 | 0 | 4 | 1 | 4 |
| 2 | 23 | 21 | 15 | 13 | 14 | 13 | 17 | 14 |
| 3 | 35 | 32 | 25 | 22 | 25 | 22 | 27 | 24 |
| 4 | 46 | 41 | 35 | 30 | 34 | 32 | 36 | 32 |
| 6 | 64 | 62 | 53 | 46 | 51 | 49 | 51 | 47 |
| 8 | 73 | 74 | 60 | 58 | 60 | 61 | 64 | 64 |
| 10 | 74 | 79 | 64 | 67 | 65 | 68 | 64 | 72 |
| 12 | 75 | 82 | 65 | 71 | 68 | 71 | 67 | 75 |
| 14 | 76 | 83 | 66 | 71 | 71 | 72 | 69 | 77 |
| 16 | 78 | 82 | 67 | 72 | 71 | 74 | 71 | 79 |
| 18 | 79 | 84 | 69 | 76 | 71 | 79 | 73 | 76 |
| 20 | 79 | 83 | 68 | 75 | 72 | 79 | 74 | 76 |
| 24 | 81 | 83 | 70 | 75 | 73 | 85 | 76 | 75 |

| | | | | |
|---|---|---|---|---|
| F2 | 68 | 65 | 65 | 65 |

Comparative dissolution profile of Tegretol XR 400 mg (represented as TG in the table 5) and formulation L of 400 mg in CDP Multimedia 1800 ml, Apparatus USP Type I (Basket), 50 RPM, at 37±0.5° C., is determined and the dissolution profile is provided in Table 6 (herein below) and a graphical representation of the same is provided in FIG. 6.

TABLE 6

| | Formulation | | | | | | | |
|---|---|---|---|---|---|---|---|---|
| | TG | L | TG | L | TG | L | TG | L |
| | Media | | | | | | | |
| | | | | | pH 4.5 | | pH 6.8 | |
| | | | | | Acetate | | Phosphate | |
| | Water | | 0.1N HCl | | buffer | | buffer | |
| | Units | | | | | | | |
| | n = 6 | n = 6 | n = 6 | n = 6 | n = 6 | n = 6 | n = 6 | n = 6 |
| | Condition | | | | | | | |
| | 900 ml, USP Type I (Basket), 50 RPM | | | | | | | |
| Time | Cumulative % Drug Released | | | | | | | |
| 1 | 6 | 5 | 4 | 3 | 2 | 1 | 6 | 9 |
| 2 | 16 | 16 | 12 | 13 | 9 | 9 | 16 | 18 |
| 3 | 28 | 26 | 23 | 21 | 18 | 18 | 26 | 28 |
| 4 | 39 | 37 | 30 | 28 | 27 | 25 | 32 | 36 |
| 6 | 55 | 57 | 49 | 42 | 43 | 41 | 47 | 51 |
| 8 | 66 | 72 | 63 | 55 | 59 | 56 | 55 | 66 |
| 10 | 71 | 77 | 71 | 61 | 70 | 69 | 61 | 74 |
| 12 | 73 | 80 | 75 | 63 | 76 | 73 | 66 | 80 |
| 14 | 73 | 81 | 78 | 65 | 78 | 73 | 65 | 79 |
| 16 | 75 | 81 | 79 | 65 | 79 | 74 | 67 | 85 |
| 18 | 75 | 81 | 80 | 66 | 80 | 75 | 71 | 87 |
| 20 | 75 | 81 | 80 | 67 | 81 | 75 | 71 | 85 |
| 24 | 75 | 81 | 81 | 68 | 81 | 75 | 71 | 85 |

| | | | | |
|---|---|---|---|---|
| F2 | 64 | 50 | 71 | 47 |

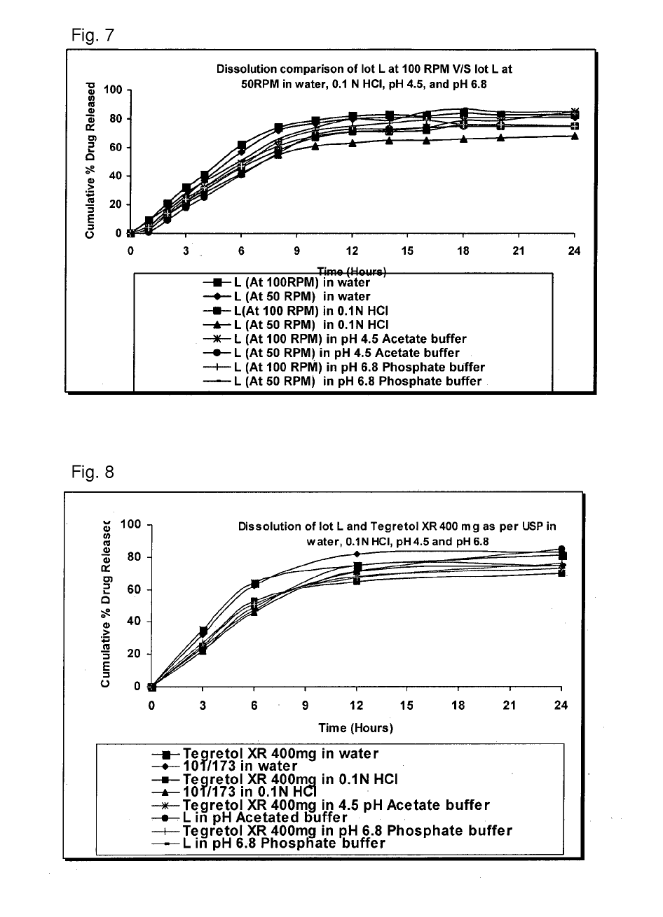

Comparative dissolution profile of formulation L at 100 RPM V/S formulation L at 50 RPM in CDP Multimedia 1800 ml, Apparatus USP Type 1 (Basket), at 37±0.5° C., is determined and the dissolution profile is provided in Table 7 (herein below) and a graphical representation of the same is provided in FIG. 7.

TABLE 7

| | Formulation | | | | | | | |
|---|---|---|---|---|---|---|---|---|
| | L | L | L | L | L | L | L | L |
| | RPM | | | | | | | |
| | 100 | 50 | 100 | 50 | 100 | 50 | 100 | 50 |
| | Media | | | | | | | |
| | | | | | pH 4.5 | | pH 6.8 | |
| | | | | | Acetate | | Phosphate | |
| | Water | | 0.1N HCl | | buffer | | buffer | |
| | Units | | | | | | | |
| | n = 6 | n = 6 | n = 6 | n = 6 | n = 6 | n = 6 | n = 6 | n = 6 |
| | Condition | | | | | | | |
| | 1800 ml, USP Type I (Basket), 100 RPM | | | | | | | |
| Time | Cumulative % Drug Released | | | | | | | |
| 0 | 0 | 0 | 0 | 0 | 0 | 0 | 0 | 0 |
| 1 | 9 | 5 | 4 | 3 | 4 | 1 | 4 | 9 |
| 2 | 21 | 16 | 13 | 13 | 13 | 9 | 14 | 18 |
| 3 | 32 | 26 | 22 | 21 | 22 | 18 | 24 | 28 |
| 4 | 41 | 37 | 30 | 28 | 32 | 25 | 32 | 36 |
| 6 | 62 | 57 | 46 | 42 | 49 | 41 | 47 | 51 |
| 8 | 74 | 72 | 58 | 55 | 61 | 56 | 64 | 66 |
| 10 | 79 | 77 | 67 | 61 | 68 | 69 | 72 | 74 |
| 12 | 82 | 80 | 71 | 63 | 71 | 73 | 75 | 80 |
| 14 | 83 | 81 | 71 | 65 | 72 | 73 | 77 | 79 |
| 16 | 82 | 81 | 72 | 65 | 74 | 74 | 79 | 85 |
| 18 | 84 | 81 | 76 | 66 | 79 | 75 | 76 | 87 |
| 20 | 83 | 81 | 75 | 67 | 79 | 75 | 76 | 85 |
| 24 | 83 | 81 | 75 | 68 | 85 | 75 | 75 | 85 |

| | | | | |
|---|---|---|---|---|
| F2 | 72 | 62 | 65 | 61 |

Comparative dissolution profile of Tegretol XR 400 mg (represented as TG in the table 5) and formulation L of 400 mg at 100 RPM as per USP in CDP Multimedia 1800 ml, Apparatus USP Type I (Basket), at 37±0.5° C., is determined and the dissolution profile is provided in Table 8 (herein below) and a graphical representation of the same is provided in FIG. 8.

TABLE 8

| | Formulation | | | | | | | |
|---|---|---|---|---|---|---|---|---|
| | TG | L | TG | L | TG | L | TG | L |
| | Media | | | | | | | |
| | | | | | pH 4.5 | | pH 6.8 | |
| | | | | | Acetate | | Phosphate | |
| | Water | | 0.1N HCl | | buffer | | buffer | |
| | Units | | | | | | | |
| | n = 6 | n = 6 | n = 6 | n = 6 | n = 6 | n = 6 | n = 6 | n = 6 |
| | Condition | | | | | | | |
| | 1800 ml, USP Type I (Basket), 100 RPM | | | | | | | |
| Time | Cumulative % Drug Released | | | | | | | |
| 0 | 0 | 0 | 0 | 0 | 0 | 0 | 0 | 0 |
| 3 | 35 | 32 | 25 | 22 | 25 | 22 | 27 | 24 |
| 6 | 64 | 62 | 53 | 46 | 51 | 49 | 51 | 47 |
| 12 | 75 | 82 | 65 | 71 | 68 | 71 | 67 | 75 |
| 24 | 81 | 83 | 70 | 75 | 73 | 85 | 76 | 75 |

| | | | | |
|---|---|---|---|---|
| F2 | 71 | 65 | 61 | 67 |

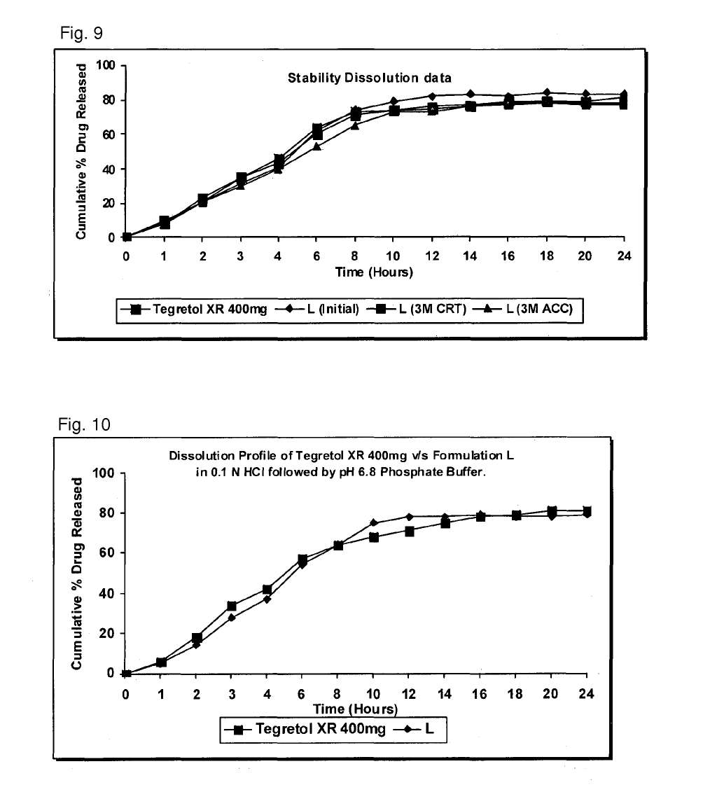

Comparative stability dissolution profile of Tegretol XR 400 mg and formulation L at 100 RPM, 1800 ml water, Apparatus USP Type I (Basket), at 37±0.5° C., the stability dissolution profile is determined and is provided in Table 9 (herein below) and a graphical representation of the same is provided in FIG. 9.

TABLE 9

| | Formulation | | | |
|---|---|---|---|---|
| | Tegretol XR | | L | L |
| | 400 mg | L | (3M CRT) | (3M ACC) |
| Units | n = 6 | n = 6 | n = 6 | n = 6 |
| Condition | Water, 1800 ml, USP Type I (Basket), 100 RPM | | | |
| Time | Cumulative % Drug Released | | | |
| 0 | 0 | 0 | 0 | 0 |
| 1 | 8 | 9 | 10 | 8 |
| 2 | 23 | 21 | 21 | 21 |
| 3 | 35 | 32 | 35 | 30 |
| 4 | 46 | 41 | 43 | 40 |
| 6 | 64 | 62 | 60 | 53 |
| 8 | 73 | 74 | 71 | 65 |
| 10 | 74 | 79 | 74 | 73 |
| 12 | 75 | 82 | 76 | 73 |
| 14 | 76 | 83 | 77 | 76 |
| 16 | 78 | 82 | 79 | 77 |
| 18 | 79 | 84 | 79 | 78 |
| 20 | 79 | 83 | 78 | 77 |
| 24 | 81 | 83 | 78 | 77 |

| | | | |
|---|---|---|---|
| F2 | 68 | 86 | 70 |

Comparative Dissolution Profile of Tegretol XR 400 mg v/s Formulation L in 0.1 N HCl followed by pH 6.8 Phosphate Buffer is determined and is provided in Table 10 (herein below) and a graphical representation of the same is provided in FIG. 10.

TABLE 10

| | Formulation | | |
|---|---|---|---|
| | Media | Tegretol XR | L |
| Units | | n = 6 | n = 6 |
| Cond | 1800 ml, USP Type I (Basket), 100 RPM | | |
| Time | | | |
| 1 | 0.1N | 6 | 5 |
| 2 | | 18 | 14 |
| 3 | pH 6.8 | 34 | 28 |
| 4 | | 42 | 37 |
| 6 | | 57 | 54 |
| 8 | | 64 | 64 |
| 10 | | 68 | 75 |
| 12 | | 71 | 78 |
| 14 | | 75 | 78 |
| 16 | | 78 | 79 |
| 18 | | 79 | 78 |
| 20 | | 81 | 78 |
| 24 | | 81 | 79 |

| | |
|---|---|
| F2 | 78 |

Linear mean plot of Carbamazepine plasma concentration Vs Time for all subjects under fed condition (n=13) is determined and a Graphical presentation of the same is provided in FIG. 11.

1. An extended release pharmaceutical composition comprising:
a. a matrix core comprising Carbamazepine or a pharmaceutically acceptable salt thereof as an active ingredient, and one or more pharmaceutically acceptable excipient(s) and;
b. a coating comprising at least one hydrophobic release controlling agent and at least one hydrophilic release controlling agent.

2. An extended release pharmaceutical composition according to claim 1 wherein Carbamazepine or a pharmaceutically acceptable salt thereof is present from about 1% to about 80% by weight of total weight of composition.

3. An extended release pharmaceutical composition according to claim 1 wherein Carbamazepine or a pharmaceutically acceptable salt thereof is present from about 5% to about 65% by weight of total weight of composition.

4. An extended release pharmaceutical composition according to claim 1 wherein the ratio of the at least one hydrophobic release controlling agent to the at least one hydrophilic release controlling agent is between 0.1:10 to 10:0.1.

5. An extended release pharmaceutical composition according to claim 1 wherein the coating comprises from about 0.1% to about 50% w/w of the core.

6. An extended release pharmaceutical composition according to claim 1 wherein the coating comprises from about 0.5% to about 20% w/w of the core.

7. An extended release pharmaceutical composition according to claim 1 wherein core tablets can be prepared by wet granulation, dry granulation, melt granulation and the like.

8. A coated extended release pharmaceutical composition according to claim 1 wherein the hydrophobic release controlling agent is selected from the group consisting of polyvinyl acetate dispersion; ethyl cellulose; cellulose acetate; low, medium or high molecular weight cellulose propionate; cellulose acetate propionate; cellulose acetate butyrate; cellulose acetate phthalate; cellulose triacetate; poly(methyl methacrylate); poly(ethyl methacrylate); poly(butyl methacrylate); poly(isobutyl methacrylate); poly(hexyl methacrylate); poly (isodecyl methacrylate); poly(lauryl methacrylate); poly(phenyl methacrylate); poly(methyl acrylate), poly(isopropyl acrylate); poly(isobutyl acrylate); poly (octadecyl acrylate); a wax, including but not limited to beeswax, carnauba wax, paraffin wax, microcrystalline wax, or ozokerite; a fatty alcohol including but not limited to cetostearyl alcohol, stearyl alcohol, cetyl alcohol and myristyl alcohol; a fatty acid ester, including but not limited to glyceryl monostearate; glycerol monooleate, acetylated monoglycerides, tristearin, tripalmitin, cetyl esters wax, glyceryl palmitostearate, glyceryl behenate, hydrogenated vegetable oils and the like.

9. A coated extended release pharmaceutical composition according to claim 1 wherein the hydrophilic release controlling agent is selected from the group consisting of a water soluble polymer, including but not limited to hydroxyethyl cellulose, hydroxypropyl cellulose, carboxymethyl cellulose, hydroxypropylmethylcellulose, sodium carboxymethyl cellulose, vinylpyrrolidone/vinyl acetate copolymer, polyvinyl alcohol, polyethylene glycol and the like; a Saccharide; including but not limited to monosaccharides, disaccharides, oligosaccharides or polysaccharides; a sugar alcohol, including but not limited to sucrose, xylitol, mannitol, sorbitol, glucose, fructose, galactose, maltitol, lactose and maltodextrin; Water soluble organic acids; water soluble salts of organic acids; water soluble organic bases; water soluble salts of organic bases, including but not limited to citric acid or salts thereof; amino acids or salts thereof; and inorganic salts, including but not limited to sodium carbonate, sodium bicarbonate, potassium chloride and sodium chloride and the like.

10. A coated extended release pharmaceutical composition comprising an immediate release core containing Carbamazepine or a pharmaceutically acceptable salt thereof and one or more pharmaceutical excipient(s) wherein the core is coated with coating comprising a rate controlling composition comprising one or more hydrophobic agent(s) and one or more hydrophilic agent(s).

11. An extended release pharmaceutical composition according to claim 10 wherein Carbamazepine or the pharmaceutically acceptable salt thereof is present from about 1% to about 80% by weight of total weight of composition.

12. An extended release pharmaceutical composition according to claim 10 wherein Carbamazepine or the pharmaceutically acceptable salt thereof is present from about 5% to about 65% by weight of total weight of composition.

13. An extended release pharmaceutical composition according to claim 10 wherein the ratio of the one or more hydrophobic agent(s) to the one or more hydrophilic agent(s) is between 0.1:10 to 10:0.1.

14. An extended release pharmaceutical composition according to claim 10 wherein the coating comprises from about 0.1% to about 50% w/w of the core.

15. An extended release pharmaceutical composition according to claim 10 wherein the coating comprises from about 0.5% to about 20% w/w of the core.

16. An extended release pharmaceutical composition according to claim 10 wherein core tablets can be prepared by wet granulation, dry granulation, melt granulation and the like.

17. A coated extended release pharmaceutical composition according to claim 10 wherein the one or more hydrophobic agent(s) is/are selected from the group consisting of polyvinyl acetate dispersion; ethyl cellulose; cellulose acetate; low, medium or high molecular weight cellulose propionate; cellulose acetate propionate; cellulose acetate butyrate; cellulose acetate phthalate; cellulose triacetate; poly(methyl methacrylate); poly(ethyl methacrylate); poly(butyl methacrylate); poly(isobutyl methacrylate); poly(hexyl methacrylate); poly(isodecyl methacrylate); poly(lauryl methacrylate); poly(phenyl methacrylate); poly(methyl acrylate), poly(isopropyl acrylate); poly(isobutyl acrylate); poly(octadecyl acrylate); a wax, including but not limited to beeswax, carnauba wax, paraffin wax, microcrystalline wax, or ozokerite; a fatty alcohol including but not limited to cetostearyl alcohol, stearyl alcohol, cetyl alcohol and myristyl alcohol; and a fatty acid ester, including but not limited to glyceryl monostearate; glycerol monooleate, acetylated monoglycerides, tristearin, tripalmitin, cetyl esters wax, glyceryl palmitostearate, glyceryl behenate, hydrogenated vegetable oils and the like.

18. A coated extended release pharmaceutical composition according to claim 10 wherein the one or more hydrophilic agent is/are selected from the group consisting of a water soluble polymer, including but not limited to hydroxyethyl cellulose, hydroxypropyl cellulose, carboxymethyl cellulose, hydroxypropylmethylcellulose, sodium carboxymethyl cellulose, vinylpyrrolidone/vinyl acetate copolymer, polyvinyl alcohol, polyethylene glycol and the like; a Saccharide; including but not limited to monosaccharides, disaccharides, oligosaccharides or polysaccharides a sugar alcohol, including but not limited to sucrose, xylitol, mannitol, sorbitol, glucose, fructose, galactose, maltitol, lactose and maltodextrin; Water soluble organic acids; water soluble salts of organic acids; water soluble organic bases; water soluble salts of organic bases, including but not limited to citric acid or salts thereof; amino acids or salts thereof; and inorganic salts, including but not limited to sodium carbonate, sodium bicarbonate, potassium chloride and sodium chloride and the like.

19. An extended release pharmaceutical composition configured for once daily dosing comprising a core containing Carbamazepine or a pharmaceutically acceptable salt, derivative, prodrug, metabolite and polymorph thereof and a pharmaceutically acceptable excipient having a in-vitro dissolution rate when measured using the USP Type I (Basket apparatus) at 100 rpm in 1800 mL, 0.1N hydrochloric acid for first 2 hours followed by the media with pH 6.8 phosphate buffer at 37° C.±0.5° C., and a coating comprising a rate controlling composition comprising one or more hydrophobic agent(s) and one or more hydrophilic agent(s), wherein the composition is configured to release:
a. from about 5 to about 25% Carbamazepine after 1 hour;
b. from about 10 to about 45% Carbamazepine after 4 hours;
c. from about 35 to about 70% Carbamazepine after 8 hours;
d. from about 55 to about 78% Carbamazepine after 12 hours;
e. from about 70 to about 78% Carbamazepine after 16 hours; or
f. greater than 78% Carbamazepine after 24 hours.

20. An extended release pharmaceutical composition according to claim 19 wherein Carbamazepine or the pharmaceutically acceptable salt thereof is present from about 1% to about 80% by weight of total weight of composition.

21. An extended release pharmaceutical composition according to claim 19 wherein Carbamazepine or the pharmaceutically acceptable salt thereof is present from about 5% to about 65% by weight of total weight of composition.

22. An extended release pharmaceutical composition according to claim 19 wherein the ratio of the one or more hydrophobic agent to hydrophilic agent is between 0.1:10 to 10:0.1.

23. An extended release pharmaceutical composition according to claim 19 wherein the coating comprises from about 0.1% to about 50% w/w of the core.

24. An extended release pharmaceutical composition according to claim 19 wherein the coating comprises from about 0.5% to about 20% w/w of the core.

25. An extended release pharmaceutical composition according to claim 19 wherein core tablets can be prepared by wet granulation, dry granulation, melt granulation and the like.

26. A coated extended release pharmaceutical composition according to claim 19 wherein the one or more hydrophobic agent(s) is/are selected from the group consisting of polyvinyl acetate dispersion; ethyl cellulose; cellulose acetate; low, medium or high molecular weight cellulose propionate; cellulose acetate propionate; cellulose acetate butyrate; cellulose acetate phthalate; cellulose triacetate; poly(methyl methacrylate); poly(ethyl methacrylate); poly(butyl methacrylate); poly(isobutyl methacrylate); poly(hexyl methacrylate); poly(isodecyl methacrylate); poly(lauryl methacrylate); poly(phenyl methacrylate); poly(methyl acrylate), poly(isopropyl acrylate); poly(isobutyl acrylate); poly(octadecyl acrylate); a wax, including but not limited to beeswax, carnauba wax, paraffin wax, microcrystalline wax, or ozokerite; a fatty alcohol including but not limited to cetostearyl alcohol, stearyl alcohol, cetyl alcohol and myristyl alcohol; a fatty acid ester, including but not limited to glyceryl monostearate; glycerol monooleate, acetylated monoglycerides, tristearin, tripalmitin, cetyl esters wax, glyceryl palmitostearate, glyceryl behenate, hydrogenated vegetable oils and the like.

27. A coated extended release pharmaceutical composition according to claim 19 wherein the one or more hydrophilic agent is/are selected from the group consisting of a water soluble polymer, including but not limited to hydroxyethyl cellulose, hydroxypropyl cellulose, carboxymethyl cellulose, hydroxypropylmethylcellulose, sodium carboxymethyl cellulose, vinylpyrrolidone/vinyl acetate copolymer, polyvinyl alcohol, polyethylene glycol and the like; a Saccharide; including but not limited to monosaccharides, disaccharides, oligosaccharides or polysaccharides; a sugar alcohol, including but not limited to sucrose, xylitol, mannitol, sorbitol, glucose, fructose, galactose, maltitol, lactose and maltodextrin; Water soluble organic acids water soluble salts of organic acids; water soluble organic bases; water soluble salts of organic bases, including but not limited to citric acid or salts thereof; amino acids or salts thereof; and inorganic salts, including but not limited to sodium carbonate, sodium bicarbonate, potassium chloride and sodium chloride and the like.

28. An extended release pharmaceutical composition comprising:
a. a matrix core comprising 50-1000 mg of Carbamazepine or a pharmaceutically acceptable salt thereof, one or more pharmaceutical excipients and;
b. a coating comprising at least one hydrophobic release controlling agent and at least one hydrophilic release controlling agent.

29. An extended release pharmaceutical composition according to claim 28 wherein Carbamazepine or the pharmaceutically acceptable salt thereof is present from about 1% to about 80% by weight of total weight of composition.

30. An extended release pharmaceutical composition according to claim 28 wherein Carbamazepine or the pharmaceutically acceptable salt thereof is present from about 5% to about 65% by weight of total weight of composition.

31. An extended release pharmaceutical composition according to claim 28 wherein the ratio of the at least one hydrophobic release controlling agent to the at least one hydrophilic release controlling agent is between 0.1:10 to 10:0.1.

32. An extended release pharmaceutical composition according to claim 28 wherein the coating comprises from about 0.1% to about 50% w/w of the core.

33. An extended release pharmaceutical composition according to claim 28 wherein the coating comprises from about 0.5% to about 20% w/w of the core.

34. An extended release pharmaceutical composition according to claim 28 wherein core tablets can be prepared by wet granulation, dry granulation, melt granulation and the like.

35. A coated extended release pharmaceutical composition according to claim 28 wherein the at least one hydrophobic release controlling agent(s) is/are selected from the group consisting of polyvinyl acetate dispersion; ethyl cellulose; cellulose acetate; low, medium or high molecular weight cellulose propionate; cellulose acetate propionate; cellulose acetate butyrate; cellulose acetate phthalate; cellulose triacetate; poly(methyl methacrylate); poly(ethyl methacrylate); poly(butyl methacrylate); poly(isobutyl methacrylate); poly(hexyl methacrylate); poly (isodecyl methacrylate); poly(lauryl methacrylate); poly(phenyl methacrylate); poly(methyl acrylate), poly(isopropyl acrylate); poly(isobutyl acrylate); poly (octadecyl acrylate); a wax, including but not limited to beeswax, carnauba wax, paraffin wax, microcrystalline wax, or ozokerite; a fatty alcohol including but not limited to cetostearyl alcohol, stearyl alcohol, cetyl alcohol and myristyl alcohol; a fatty acid ester, including but not limited to glyceryl monostearate; glycerol monooleate, acetylated monoglycerides, tristearin, tripalmitin, cetyl esters wax, glyceryl palmitostearate, glyceryl behenate, hydrogenated vegetable oils and the like.

36. A coated extended release pharmaceutical composition according to claim 28 wherein the at least one hydrophilic release controlling agent(s) is selected from the group consisting of a water soluble polymer, including but not limited to hydroxyethyl cellulose, hydroxypropyl cellulose, carboxymethyl cellulose, hydroxypropylmethylcellulose, sodium carboxymethyl cellulose, vinylpyrrolidone/vinyl acetate copolymer, polyvinyl alcohol, polyethylene glycol and the like; a Saccharide; including but not limited to monosaccharides, disaccharides, oligosaccharides or polysaccharides; a sugar alcohol, including but not limited to sucrose, xylitol, mannitol, sorbitol, glucose, fructose, galactose, maltitol, lactose and maltodextrin; Water soluble organic acids; water soluble salts of organic acids; water soluble organic bases; water soluble salts of organic bases, including but not limited to citric acid or salts thereof; amino acids or salts thereof; and inorganic salts, including but not limited to sodium carbonate, sodium bicarbonate, potassium chloride and sodium chloride and the like.
